(19) United States
(12) Patent Application Publication
(10) Pub. No.: US 2022/0249194 A1
(43) Pub. Date: Aug. 11, 2022

(54) A MEDICAL RETRIEVAL DEVICE

(71) Applicant: LINA MEDICAL INTERNATIONAL OPERATIONS AG, Root D4 (CH)

(72) Inventor: Kai HARREKILDE-PETERSEN, Charlottenlund (DK)

(21) Appl. No.: 17/616,534

(22) PCT Filed: Jun. 19, 2020
(86) PCT No.: PCT/EP2020/067098

§ 371 Date: Dec. 3, 2021

(30) Foreign Application Priority Data
Jun. 21, 2019 (EP) 19181724.6

(51) Int. Cl. A61B 90/00; A61B 1/05; A61B 17/221

(52) U.S. Cl. CPC A61B 90/361; A61B 1/05; A61B 2017/4216; A61B 2017/2212; A61B 17/221

(57) ABSTRACT

A medical retrieval device includes an elongated hollow shaft extending from a proximal shaft end to a distal shaft end, and an operating element forming a proximal end and a distal end and being movable in the hollow shaft between a retracted position and an extended position. The medical retrieval device further comprises a plurality of basket-wires extending from a proximal wire end to a distal wire end, where the basket-wires are joined at the distal wire end and are fixed at the proximal wire end to the distal end of the operating element. The distance L1 from the distal shaft end of the hollow shaft to the proximal wire end of the basket-wires is smaller than the distance L2 from the proximal wire end of the basket-wires to the distal wire end of the basket-wires, when the operating element is in the extended position.

### FIELD OF DISCLOSURE

The present disclosure relates to a medical retrieval device having an elongated hollow shaft extending in an axial direction from a proximal shaft end to a distal shaft end. An operating element forming a proximal end and a distal end is movable in the hollow shaft between a retracted position and an extended position. A plurality of basket-wires extending from a proximal wire end to a distal wire end and being joined at the distal wire end is, at the proximal wire end, fixed to the distal end of the operating element. Particularly, the disclosure relates to a medical retrieval device for use in hysteroscopy, cystoscopy and similar procedures.

### BACKGROUND

Gynecologists and urologists use different methods and devices for removal and retrieval of tissue e.g. from the endometrium, urethra, or bladder. A common type of device for retrieval of tissue is known as a basket. This type of device is made of multiple filaments forming a basket, and being retractable into a shaft to thereby cut tissue with the filaments and for using the reduced distance between the filaments for capturing tissue.

Hysteroscopy and cystoscopy are standard examination procedures where an endoscope is inserted into the uterus or a urinary tract e.g. for inspection and control during retrieval of tissue, e.g. polyps and fibroids, etc.

Precise positioning of endoscopes is required during the examination procedures which may contravene good ergonomic working conditions for the practitioners. Further hindrance and complication may arise when working with multiple tools such as combinations between retrieval devices and hysteroscopy or cystoscopy. In the following, whenever reference is made to either hysteroscopy or cystoscopy, the reference could be either one of the procedures.

### DESCRIPTION OF THE INVENTION

It is an object of the invention to improve preciseness, sterility, safety of operation, and simplicity of devices for hysteroscopy, cystoscopy and similar procedures.

The invention, in a first aspect, provides a medical retrieval device comprising an elongated hollow shaft extending from a proximal shaft end to a distal shaft end, an operating element forming a proximal end and a distal end and being movable in the hollow shaft between a retracted position and an extended position, and a plurality of basket-wires extending from a proximal wire end to a distal wire end, the basket-wires being joined at the distal wire end and being fixed at the proximal wire end to the distal end of the operating element, wherein the distance L1 from the distal shaft end of the hollow shaft to the proximal wire end of the basket-wires is smaller than the distance L2 from the proximal wire end of the basket-wires to the distal wire end of the basket-wires, when the operating element is in the extended position.

The operating element is movable in the hollow shaft between a retracted position and an extended position. In the retracted position the basket-wires which are fixed to the distal end of the operating element may be fully covered by the hollow shaft and thus protected by the shaft. When a part of the hollow shaft has been inserted e.g. into a patient's uterus, the operating element may be moved in the hollow shaft to the extended position where the basket-wires are free of the hollow shaft.

Thus, in the retracted position, the distal shaft end may form the end portion of the medical retrieval device, whereas the distal wire end may form the end portion of the medical retrieval device in the extended position. The practitioner may operate the medical retrieval device by movement of a handle relative to a grip, where the grip is attached to the proximal shaft end and the handle is attached to the operating element.

The plurality of basket-wires extending from a proximal wire end to a distal wire end and joined at the distal wire end could be constituted by individual wire pieces which are joined, e.g. by knots or glue etc. It could also be constituted by a single or a few wire segments extending towards the distal end where they bend and extend backwards. Such segments could be joined at the distal wire end, e.g. by a knot or glue.

The plurality of basket-wires being fixed at the proximal wire end to the distal end of the operating element could be basket wires joined to the operating element by knots, glue or otherwise, or it could be one single wire forming both a basket wire and the operating element.

The basket-wires may comprise two or more wires, each being attached to the distal end of the operating element at both ends whereby each wire may form a loop substantially at a middle portion of the wire. At the loop which may form the distal wire end, the basket-wires are joined, e.g. by a knot or simply by coiling the wires into joined coils.

In an alternative embodiment, the basket-wires extend between opposite ends, one being joined to the operating element and the other being joined to the other basket-wires at the distal wire end by use of an adhesive or by use of a separate joining element being clamped around the basket-wires at the distal wire end to thereby form a joint of the wires at the distal wire end.

Each wire may have a diameter in the range of 0.1-0.5 mm. The basket-wires may be made of a flexible material, such as nitinol, titanium, and similar kinds of steel having the ability to return to the original shape after having been deformed elastically. Such a material allows the basket-wires to be retracted into the hollow shaft in a collapsed configuration. Movement of the operating element in the hollow shaft towards the retracted position may move the basket-wires into the hollow shaft, whereby the basket-wires may be collapsed by contact between an inner surface of the hollow shaft and the wires to thereby bring them into the collapsed configuration.

The flexible material may be a shape memory alloys allowing the basket-wires to unfold to an expanded configuration by recovering their original shape, when the operating element is moved to the extended position, where the basket-wires are free of the hollow shaft. In the expanded configuration, the basket-wires may form a space between the wires; i.e. an open basket.

In the expanded configuration, the space formed by the basket-wires may have a diameter in the range of 10-25 mm, such as in the range of 12-20 mm, and a length in the range of 20-40 mm, such as 25-35 mm, where the length equals the distance L2 from the proximal wire end to the distal wire end, and where the diameter is the dimension of the space in a direction perpendicular to the longitudinal direction of the medical retrieval device, and thus perpendicular to the length.

When inserted e.g. into a patient's uterus, the basket-wires are moved to in the expanded configuration in which they may be arranged around e.g. a polyp or a fibroid which should be removed, so that the polyp or fibroid is in the space between the wires. When moving the operating element toward the retracted position, the polyp/fibroid may be cut free by the basket-wires and may be retracted together with the operating element and hollow shaft.

By providing the distance L1 from the distal shaft end of the hollow shaft to the proximal wire end of the basket-wires is smaller than the distance L2 from the proximal wire end of the basket-wires to the distal wire end of the basket-wires, when the operating element is in the extended position, operation of the medical retrieval device may be facilitated, as a smaller distance may increase preciseness of operation of the medical retrieval device and reduce the risk of bending of the operating element extending out of the hollow shaft. The L1 which is shorter, brings the basket closer to the elongated hollow shaft which, due to its larger dimension transverse to the axial direction can be rigid relative to the operating element.

In one embodiment, the distance L1 may be less than 10% of the distance L2 thereby further facilitating operation of the medical retrieval device. If the operating element is more flexible than the hollow shaft, the hollow shaft may protect the operating element by forming a shield around it, and it may thus be an advantage that the distance L1 is smaller than the distance L2, such as less than 10% of the distance L2 to thereby increase the protected part of the operating element. In one embodiment, the distance L1 may be even smaller, such as less than 5% of the distance L2.

The operating element may be rotatably arranged in the hollow shaft. This may allow a practitioner to rotate the basket-wires via the proximal end of the operating element, e.g. by use of a handle attached hereto. Rotation of the operating element may rotate the basket-wires being fixed at the distal end of the operating element, thereby allowing correct positioning of the basket-wires relatively to e.g. a polyp in the uterus, as rotation may facilitate positioning of the space between the wires around the polyp to be retrieved.

The operating element may, alternatively, be non-rotatably arranged in hollow shaft. This may allow a practitioner to rotate the basket-wires by rotation of the hollow shaft and may improve the manoeuvrability of the basket by use of a handle attached to the hollow shaft. Since the hollow shaft could be more rigid than the operating element, the control may be less impaired by deflection. Rotation of the hollow shaft may thus rotate the operating element and thereby the basket-wires being fixed at the distal end of the operating element and correct positioning of the basket-wires relative to e.g. a polyp in the uterus may be achieved.

To avoid accumulation of unwanted material and/or to facilitate movement of the operating element in the hollow shaft, the hollow shaft may form a closed structure along the length of the elongated hollow shaft. Thus, the hollow shaft may be formed as tubular element with an opening at each of the oppositely arranged end portions and particularly, it may be formed without any openings between the open end portions. The operating element with the basket-wires may extend through the opening at the end portion at the distal end, whereas the operating element may be operated via the opposite opening at the end portion at the proximal end.

To facilitate movement of the operating element, such as movement between the retracted position and the extended position and/or rotational movement, the operating element may be more flexible than the hollow shaft. By the term ‘more flexible’ should be understood that the required force for bending the operating element is lower than the required force for bending the hollow shaft.

To achieve a small diameter of the hollow shaft, it may be an advantage that the diameter of the operating element is small, such as in the range of 0.5-2.0 mm. To achieve the required stiffness of the operating element to thereby ensure proper operation hereof, the operating element may form a second hollow shaft between the proximal end and the distal end, as an operating element in the form of a hollow shaft is less flexible than e.g. a wire and less flexible than a solid element having the same diameter. This may increase precise of operation of the medical retrieval device.

To improve overview and preciseness of the retrieval process, the medical retrieval device may further comprise a visualization device for visualization of internal tissue of a patient's uterus, the visualization device comprising a hand-held control unit, an elongated member, and an image capturing structure configured to communicate video signals with a monitor.

At least the distal end of the elongated member and the image capturing structure is dimensioned for insertion into the patient's uterus through cervix.

The elongated member forms a working channel, and the elongated hollow shaft is movable in the working channel to and from a depth limiting stop. When reaching the depth limiting stop, the hollow shaft cannot move further into the working channel.

The image capturing structure defines a depth of field (DOF) defining a distance between a nearest and the furthest objects that are in acceptably sharp focus in an image. DOF field can be calculated based on focal length, distance to subject, the acceptable circle of confusion size, and aperture.

Precise focus is only possible at an exact distance from the image capturing structure. At that distance, a point object will produce a point image. Herein we refer to this point as focal point. Focal points are all located in a specific plane referred to herein as focal plane. At all other distances, a point object will produce a blur spot shaped like the aperture, i.e. typically a circular spot. When this circular spot is so small that it becomes indistinguishable from a point, the image is considered acceptably sharp. The spot is herein referred to as acceptable circle of confusion (ACC).

In one embodiment of the invention, the image capturing structure is configured such that for a specified ACC, DOF extends from the distal shaft end to the distal wire end when the hollow shaft is at the depth limiting stop and when the operating element is in the extended position.

ACC may e.g. be specified as smaller than 0.05 mm, such as smaller than 0,04, 0,03, 0,02, or even smaller than 1, μm.

In one embodiment, the image capturing structure comprises a CCD having a specific pixel size. In this embodiment ACC may e.g. be specified to be smaller than 200% of the pixel size, such as smaller than 150%, 120%, 110% or even equal to or smaller than 100% of the pixel size. The pixel size may be smaller than 2*2 μm or even smaller than 1*1 μm. Accordingly, the ACC may be smaller than 2 μm or even smaller than 1 μm.

In one particular embodiment, the distal wire end is exactly at the focal plane when the hollow shaft is at the depth limiting stop and the operating element is in the extended position.

In another embodiment, the distal wire end is movable passed the focal plane when the hollow shaft is moved away from the depth limiting stop and the operating element is in the extended position.

By providing the working channel with a depth limiting stop for the hollow shaft, it may be achieved that the distal wire end is within the focal range or specifically at the focal point where light rays converge to form a sharp image, when the operating element is in the extended position to thereby improve quality of the displayed video signals on the monitor and thereby increase preciseness of operation of the medical retrieval device and thereby reduce risk of errors. Particularly, the combination between the depth limiting stop and the configuration of the image capturing structure for placing DOF between the distal shaft end and the distal wire end makes precise positioning of the basket relative to the image capturing structure easy and fast.

The visualization device may comprise a control unit which may be hand-held. The elongated member of the visualization device may be connectable to the control unit, and the control unit may be used without connection to any external devices, whereby the medical retrieval device including the visualisation device may become easy to use without having to assembly cables or attach external camera or monitor. This reduces the risk of errors, reduces the risk of combining non-compatible items, and reduces the risk of contaminating the device during connection to external components.

The elongated member extends along a longitudinal axis and forms a working channel for the elongated hollow shaft of the medical retrieval device. The working channel may further form space for cables and tubes inside the elongated member. Particularly, the working channel may extend along the longitudinal axis from an internal opening inside the control unit to an external opening at the distal end of the elongated member.

The image capturing structure may be movable relative to the distal end of the elongated member. This will allow better adaption of the device to the specific size and/or shape of the uterus and/or cervix of the patient and the capturing structure may e.g. be displaced during use to provide better space for the operating element with the basket-wires fixed at the distal end.

Particularly, the image capturing structure may be movable between a first position in which it at least partly hinders passage through the working channel and a second position providing unhindered passage through the working channel.

The image capturing structure may e.g. be located inside an axial bore in the distal end of the elongated member, and the bore could be made to obtain the claimed movability of the image capturing structure relative to the distal end, e.g. by providing the bore with a larger cross section than the cross section of the image capturing means such that the image capturing means can move radially relative to a longitudinal axis of the elongated member.

The axial bore may be separate from the working channel or it may form a distal end portion of the working channel. It may e.g. be constituted by a portion of the working channel where the working channel is widened out and defines an increased cross sectional area.

In one embodiment, the hollow shaft may be more flexible than the elongated member of the visualization device. This may enable the elongated shaft to adapt to the shape of the elongated member and thus allow control of the position of the basket wires by manipulation of the visualization device.

The distance L3 from the distal end of elongated member forming the working channel for the hollow shaft to the distal wire end of the basket-wires when the hollow shaft is at the depth limiting stop and the operating element is in the extended position may be less than twice the distance L2. Consequently, the elongated member may protect a substantial part of the hollow shaft. This may be particularly interesting if the hollow shaft is more flexible than the elongated member. The property of the elongated member to resist deformation may e.g. be k=F/δ where F is the force on the body and δ is the displacement produced by the force along the same degree of freedom, and k for the elongated member may be 2, 3, 4, 5 or even more than five times k for the hollow shaft.

The hollow shaft may form an enlarged section at the proximal shaft end of the hollow shaft, where the diameter of the enlarged section is larger than the diameter of the elongated member. The enlarged section may form a depth limiting member which, when reaching the elongated member forms the depth limiting stop. I.e. when the hollow shaft is inserted into the working channel of the elongated member, the enlarged section abuts the proximal end of the elongated member which is then the depth limiting stop.

The enlarged section may in one embodiment be formed by a spring element arranged circumferentially around the hollow shaft. In this embodiment, the depth limiting stop may be at the position where the spring element abuts the proximal end of the elongated member.

The use of a spring element provides the advantage that the hollow shaft may preserve the rigidity k, and that k for the hollow shaft may therefore remain being low relative to k for the elongated member, also at the enlarged section.

### DETAILED DESCRIPTION

In the following, embodiments of the invention will be described in further details with reference to the drawing, in which:

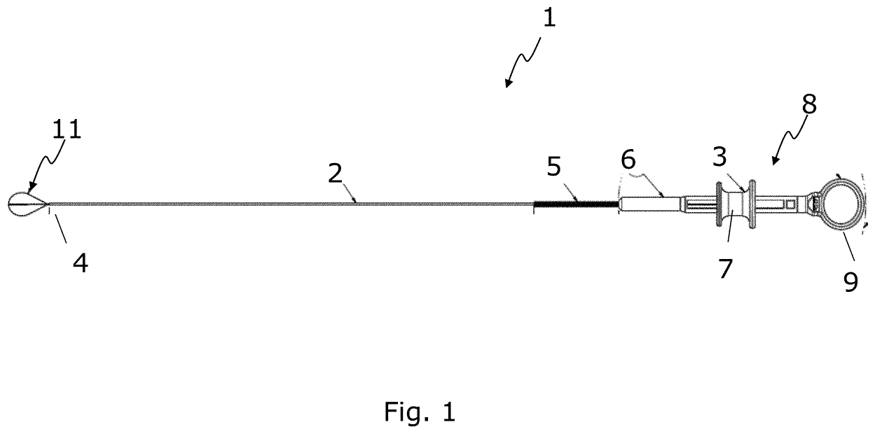

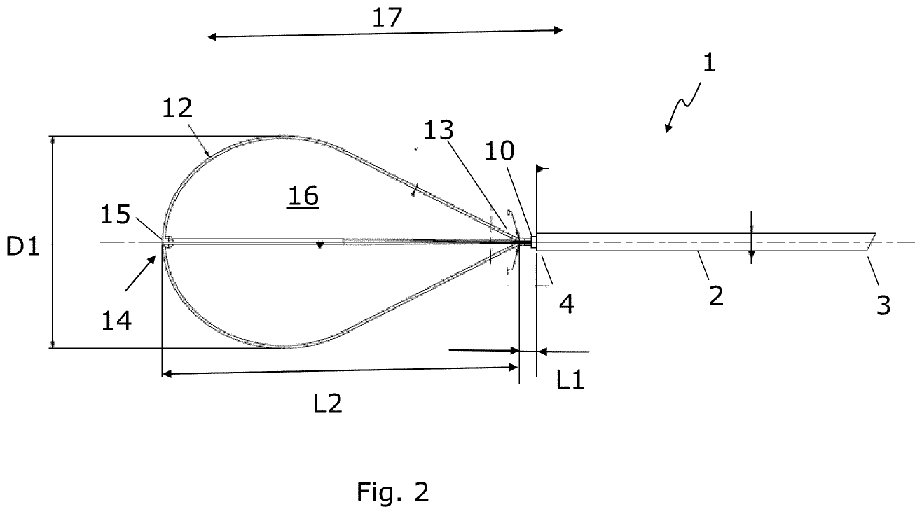

FIGS. 1-2 disclose an embodiment of a medical retrieval device,

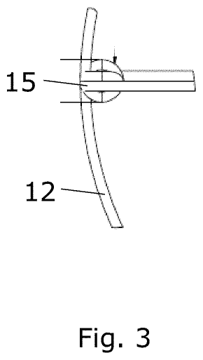

FIG. 3 discloses a distal wire end,

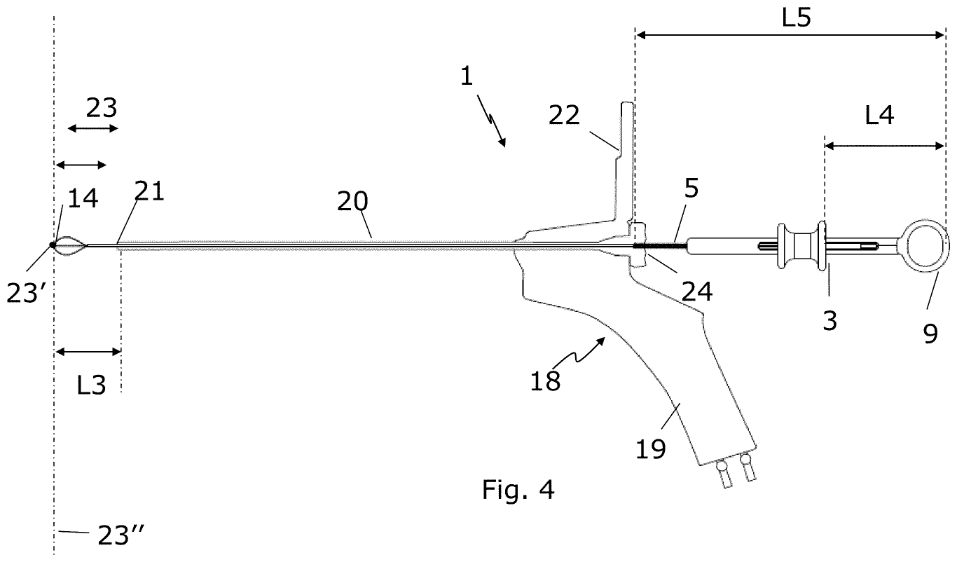

FIG. 4 discloses an embodiment of a medical retrieval device with a visualisation device, and

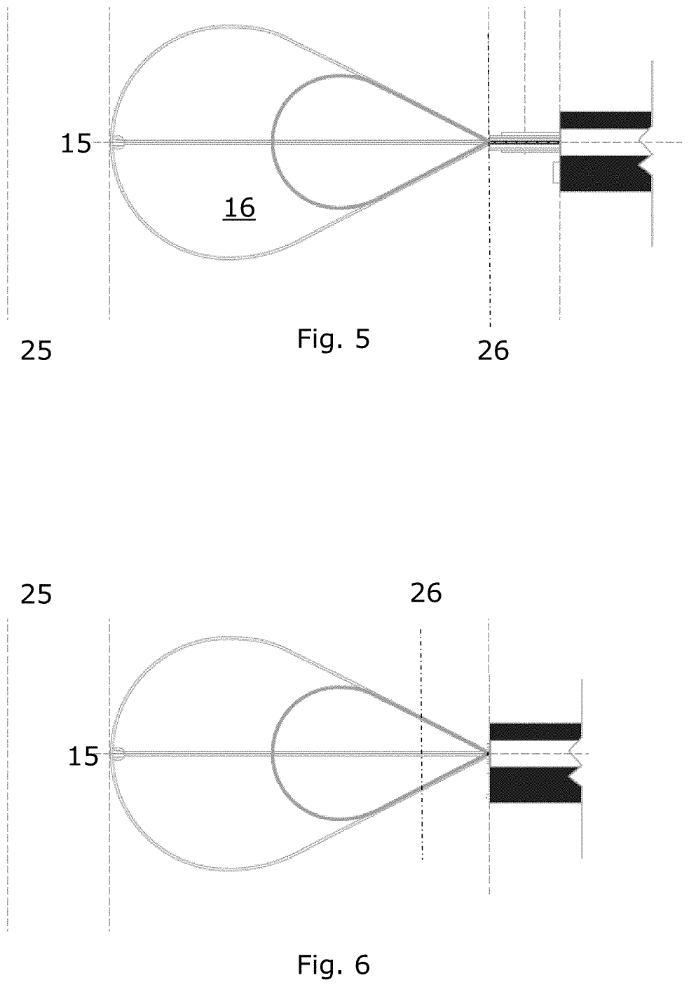

FIGS. 5 and 6 illustrate details of DOF.

It should be understood that the detailed description and specific examples, while indicating embodiment of the invention, are given by way of illustration only, since various changes and modifications within the spirit and scope of the invention will become apparent to those skilled in the art from this detailed description.

FIG. 1 discloses a medical retrieval device 1 and FIG. 2 discloses an enlarged view of a distal part of the medical retrieval device 1. The medical retrieval device 1 comprises an elongated hollow shaft 2 extending from a proximal shaft end 3 to a distal shaft end 4. In the proximal shaft end 3, the shaft widens out and defines an enlarged section 5, a handle section 6, and a hand grip 7. An operating element 8 forms a proximal end 9 which is shaped as an operating grip. The opposite, distal end 10 (c.f. FIG. 2) of the operating element 8 is attached to the basket 11. The operating element 8 is movable in the hollow shaft 2 between a retracted position and an extended position. FIGS. 1 and 2 disclose the operating element 8 in the extended position where the basket is unfolded and ready to receive tissue to be retracted.

The enlarged section 5 at the proximal shaft end 3 of the hollow shaft, where the diameter of the enlarged section 5 is decreased compared to diameter of the remaining part of the hollow shaft. Furthermore, the diameter of the enlarged section 5 may form a depth limiting function when the retrieval device is used with a visualization device. When the hollow shaft is inserted in a working channel of an elongated member of the visualization device, it can only be inserted until the point where the enlarged section 14 abuts a proximal end of the elongated member.

In the illustrated embodiment, the enlarged section 14 is formed by a spring element arranged circumferentially around the hollow shaft 2. Thus, the depth limiting stop is at the position where the spring element 14 is in a compressed configuration.

The basket 11 comprises a plurality of basket-wires 12 extending from a proximal wire end 13 to a distal wire end 15. The basket-wires 12 are joined at the distal wire end 15 by a knot and they are fixed at the proximal wire end 13 to the distal end 10 of the operating element 8.

The distance L1 from the distal shaft end 4 of the hollow shaft 2 to the proximal wire end 13 of the basket-wires 12 is smaller than the distance L2 from the proximal wire end 13 of the basket-wires 12 to the distal wire end 15 of the basket-wires 12, when the operating element 5 is in the extended position as illustrated in FIG. 1.

When the operating element 8 is in the extended position, the basket-wires 12 are an expanded configuration forming an inner space 16. In the illustrated embodiment, the space 16 formed by the basket-wires has a diameter D1 of approximately 16 mm and a length of approximately 30 mm, where the length equals the distance L2 from the proximal wire end to the distal wire end, and where the diameter D1 is the dimension of the inner space 16 in a direction perpendicular to the longitudinal direction of the medical retrieval device 1. The longitudinal direction is indicated by the arrow 17.

FIG. 3 discloses a distal wire end 15 of the medical retrieval device 1 illustrated in FIGS. 1 and 2. In the illustrated embodiment, the basket-wires 12 comprise two wires, each being attached to the distal end 10 of the operating element 8 at both ends whereby each wire 12 forms a loop substantially at a middle portion of the wire 12. At the loop which forms the distal wire end 15, the basket-wires 12 are joined by a knot at the distal wire end 15.

FIG. 4 illustrates a medical retrieval device with a visualization device 18.

The visualization device 18 comprising a hand-held control unit 19, an elongated member 20, and an image capturing structure 21 configured to communicate video signals with a monitor 22.

The image capturing structure comprises a CCD having a specific pixel size.

The image capturing structure defines a DOF 23, and a specific focal point 23′ can be identified at the focal plane 23″. At points in this plane light rays converge to form a sharp image.

The hollow shaft is inserted in the elongated member 20, i.e. the depth limiting stop defined by the enlarged section 5 has reached the proximal end 24 of the elongated member 20. In this position of the hollow shaft, the retrieval device defines five different lengths, L1, L2, L3, L4, and L5.

L1, L2, and L3 are defined above.

L4 is the distance from the proximal end 9 of the operating element to the proximal end 3 of the hollow shaft 2.

L5 is the distance from the depth limiting stop to the proximal end of the operating element.

The following condition may apply: L1<L2<L3<L4<L5.

FIGS. 5 and 6 illustrate details of DOF which extends between the far focus plane 25 and the near focus plane 26. The near and far focus planes 25, 26 are defined by the optics of the image capturing structure 21 in front of the visualization device, i.e. in front of a CCD.

In FIG. 5, the hollow shaft (2) is at the depth limiting stop and the operating element (8) is in the extended position. In FIG. 6, the hollow shaft (2) is retracted from the depth limiting stop to a position where the hollow shaft is flush with the elongated member (20). In the disclosed embodiment, the hollow shaft is thereby also flush with the image capturing structure, or the optics thereof. In FIG. 6, the operating element (8) is in the extended position.

In FIG. 5, the hollow shaft (2) when being at the depth limiting stop is at the near focus plane. In the disclosed embodiment, it is flush with the near focus plane. The distal wire end 15 is within DOF between the near and far focus planes. In the disclosed embodiment, the entire inner space 26 is within DOF.

In FIG. 6 when the hollow shaft (2) is retracted, the distal wire end 15 is still within DOF. A large part of the inner space 16 is in DOF. In the disclosed embodiment, more than 80 percent of the inner space is in DOF when the hollow shaft is flush with the image capturing structure 21.

1. A medical retrieval device comprising:
an elongated hollow shaft extending in an axial direction from a proximal shaft end to a distal shaft end,
an operating element forming a proximal end and a distal end and being movable in the hollow shaft between a retracted position and an extended position, and
a plurality of basket-wires extending from a proximal wire end to a distal wire end, the basket-wires being joined at the distal wire end and being fixed at the proximal wire end to the distal end of the operating element,
wherein the distance L1 from the distal shaft end of the hollow shaft to the proximal wire end of the basket-wires is smaller than the distance L2 from the proximal wire end of the basket-wires to the distal wire end of the basket-wires, when the operating element is in the extended position.

2. The medical retrieval device according to claim 1, wherein the distance L1 is less than 10% of the distance L2.

3. The medical retrieval device according to claim 1, wherein the operating element is non-rotatably in the hollow shaft.

4. The medical retrieval device according to claim 1, wherein the hollow shaft forms a closed structure along the length of the elongated hollow shaft.

5. The medical retrieval device according to claim 1, wherein the operating element is more flexible than the hollow shaft.

6. The medical retrieval device according to claim 1, wherein the operating element forms a second hollow shaft between the proximal end and the distal end.

7. The medical retrieval device according to claim 1, further comprising a visualization device for visualization of internal tissue of a patient's uterus, the visualization device comprising a hand-held control unit, an elongated member, and an image capturing structure configured to communicate video signals with a monitor and forming a depth of field with a focal plane where light rays converge to form a sharp image, and where at least the distal end of the elongated member and the image capturing structure is dimensioned for insertion into the patient's uterus through cervix, the elongated member forming a working channel, and the elongated hollow shaft being movable in the working channel to and from a depth limiting stop, the image capturing structure being configured, for a specific ACC to provide the DOF from the distal shaft end to the distal wire when the hollow shaft is at the depth limiting stop and when the operating element is in the extended position.

8. The medical retrieval device according to claim 7, wherein the specific ACC is smaller than 2 μm.

9. The medical retrieval device according to claim 7, wherein the image capturing structure comprises a CCD having a specific pixel size and wherein the specific ACC is smaller than 200% of the pixel size.

10. The medical retrieval device according to claim 7, wherein the distal wire end is at the focal plane when the hollow shaft is at the depth limiting stop and the operating element is in the extended position.

11. The medial retrieval device according to claim 7, wherein the distance L3 from the distal end of elongated member to the distal wire end of the basket-wires when the hollow shaft is at the depth limiting stop and the operating element is in the extended position is less than twice the distance L2.

12. The medical retrieval device according to claim 7, wherein the hollow shaft is more flexible than the elongated member of the visualization device.

13. The medical retrieval device according to claim 7, wherein the hollow shaft forms an enlarged section at the proximal shaft end of the hollow shaft, and wherein the diameter of the enlarged section is larger than the diameter of the working channel.

14. The medical retrieval device according to claim 13, wherein the enlarged section is formed by a spring element arranged circumferentially around the hollow shaft.
